(19) United States
(12) Patent Application Publication
(10) Pub. No.: US 2013/0115202 A1
(43) Pub. Date: May 9, 2013

(54) ANTI-INFLAMMATORY COMPOSITIONS FOR TREATING NEURO-INFLAMMATION

(71) Applicant: Theta Biomedical Consulting & Development Co., Inc, Brookline, MA (US)

(72) Inventor: Theoharis C. THEOHARIDES, Brookline, MA (US)

(73) Assignee: THETA BIOMEDICAL CONSULTING & DEVELOPMENT CO., INC, Brookline, MA (US)

(21) Appl. No.: 13/722,397

(22) Filed: Dec. 20, 2012

Related U.S. Application Data
(63) Continuation-in-part of application No. 13/622,246, filed on Sep. 18, 2012.
Application No. 13/622,246 is a continuation of application No. 12/861,152, filed on Aug. 23, 2010, now Pat. No. 8,268,365.
Application No. 12/861,152 is a continuation of application No. 11/214,831, filed on Aug. 31, 2005, now Pat. No. 7,906,153.

(51) Int. Cl. A61K 45/06; A61K 31/122; A61K 31/205; A61K 31/737; A61K 36/76; A61K 31/7076; A61K 31/455; A61K 36/886; A61K 35/56; A61K 31/496; A61K 31/4745; A61K 31/728; A61K 36/53; A61K 31/05; A61K 31/519; A61K 33/04; A61K 31/7048; A61K 31/4188; A61K 31/222; A61K 31/352

(52) U.S. Cl. CPC A61K 45/06; A61K 31/352; A61K 31/122; A61K 31/205; A61K 31/737; A61K 36/76; A61K 31/7076; A61K 31/455; A61K 36/886; A61K 35/57; A61K 31/496; A61K 31/4745; A61K 31/728; A61K 36/53; A61K 31/05; A61K 31/519; A61K 33/04; A61K 31/7048; A61K 31/4188; A61K 31/222
USPC 424/94.1; 514/456; 514/54; 424/775; 424/522; 514/255.04; 514/280; 424/702; 514/27

(57) ABSTRACT

This disclosure pertains to methods of treating a neuro-inflammation disorder in a subject, comprising administering to a subject in need thereof an effective amount of a composition comprising a flavonoid, or a structurally related analogue, olive kernel extract, hydroxytyrosol, and berberine, and, optionally, one or more ingredients selected from the group consisting of a sulfated proteoglycan, oleocanthal, a CRH antagonist, S adenosylmethionine, a histamine 1 receptor antagonist, a histamine 3 receptor agonist, emu oil, oregano oil, grape seed oil, aloe extract, biotin, and selenium. Certain of the present compositions are useful in protecting against or treating neuro-inflammation associated with allergies, Alzheimer's disease (AD), atherosclerosis, asthma, Autistic Spectrum Disorders (ASD).

This application is a continuation-in-part application of U.S. application Ser. No. 13/622,246, filed Sep. 18, 2012, which is a continuation of U.S. application Ser. No. 12/861,152, filed Aug. 23, 2010, now U.S. Pat. No. 8,268,365, which is a continuation of U.S. application Ser. No. 11/214,831, filed Aug. 31, 2005, now U.S. Pat. No. 7,906,153, all of which are incorporated herein by reference in their entireties.

### BACKGROUND OF THE INVENTION

The invention is generally related to the treatment of conditions and disorders associated with neuro-inflammation. More specifically, the invention is related to compositions containing inhibitors of mast cell activation and secretion of inflammatory and neurotoxic molecules in respone to neuropeptide triggers derived from the nervous system such as corticotropin-releasing hormone (CRH), myelin basic protein (MBP), neurotensin (NT) or substance P (SP), as well as flavonoid compounds that are designed to be used as dietary supplements, medical foods, botanical drugs or adjuvants to conventional approved medications for the prevention or treatment of conditions and diseases associated with neuro-inflammation e.g., in the brain as in Alzheimer's disease (AD) and Autism Spectrum Disorders (ASD).

Neuro-inflammation is heretofore defined as a physiological or pathological process involving inflammation of the peripheral or central nervous system and associated with activation of immune cells, especially mast cells, but also macrophages, microglial cells and T cells by substances derived from the brain, such as CRH, MBP, NT or SP.

Theoharides, et al., British Journal of Pharmacology 131:1039 (2000) indicated for the first time how proteoglycans, such as chondroitin sulfate, may reduce inflammation. This paper reported that chondroitin sulfate inhibits activation of mast cells that are known to trigger allergy and inflammation.

Mast cells are also now recognized as important causative intermediaries in many painful inflammatory conditions [Galli, N. Eng. J. Med. 328:257 (1993); Theoharides, Int. J. Tissue Reactions 18:1 (1996)], such as interstitial cystitis and irritable bowel syndrome [Theoharides et al., Ann. NY Acad. Sci. 840:619 (1998)], as well as in migraines [Theoharides, T. C., Brain Res. Rev. 49:65 (2005), multiple sclerosis [Theoharides, Persp Biol Med. 26:672 (1983), Theoharides, Life Sci. 46:607 (1996), and J. Neuroimmunol. 146:1 (2004) and ASD [Theoharides et al., Biochem. Biophys. Acta 1822:34 (2010) ].

It was shown that chondroitin sulfate's ability to inhibit the activation of mast cells compliments the inhibitory effects on mast cell activation of another class of naturally occurring compounds, the flavonoids [Middleton, et al., Pharm. Rev. 52:1 (2000)]. Certain plant flavones (in citrus fruit pulp, seeds, sea weed) are now recognized as anti-allergic, anti-inflammatory, anti-oxidant and cytoprotective. Only some flavonoids, especially the flavonol quercetin [Kimata, et al., Allergy 30:501(2000)], and the flavone luteolin inhibit human mast cell activation [Kempuraj et al., Br. J. Pharmacol. 155:1076 (2008)].

Use of the term “bioflavonoids” or “citrus flavonoids” in certain commercial products provides little information, and may include molecules that have detrimental effects; for example, soy contains isoflavones that have estrogen-like activity that worsens inflammatory conditions and cancer.

Applicant has described the use of antagonists of the action of CRH (also known as Corticotropin Releasing Factor) in inhibiting myocardial mast cell activation in myocardial ischemia, in treating stress-induced skin disease (U.S. Pat. No. 6,020,305) and stress-induced migraine headaches (U.S. Pat. No. 5,855,884), the contents of which are incorporated herein by reference. The synergistic effects of the compositions of the present invention that include antagonists of the actions of CRH on mast cells were not recognized at the time of the previous studies. The word “antagonists” in connection with CRH is intended herein to include any molecule that prevents the actions of CRH on target cells, and includes, but is not limited to, anti-CRH neutralizing antibodies or binding proteins, or molecules preventing the release of CRH at local sites.

Applicant has also described a method for treating patients with mast cell derived molecules-induced interstitial cystitis with certain histamine-1 receptor antagonists (U.S. Pat. No. 5,994,357). Treatment of mast cell molecules-induced migraines with histamine-3 receptor agonists is the subject of U.S. Pat. No. 5,855,884. Histamine-3 receptor agonists as pharmaceutical agents in mast cell-involved diseases are described in U.S. Pat. No. 5,831,259. Use of flavonoids in treatment of cancer was described in U.S. Pat. No. 7,799,766, and use of flavonoids in treatment of multiple sclerosis is the subject of U.S. Pat. No. 7,906,153. The contents of these patents are incorporated herein by reference. At the time of this invention the synergistic effects of the present compositions in diseases involving brain inflammation had not yet been recognized.

An important need exists for compositions for administration to human patients being treated for mast cell-related neuro-inflammatory diseases by various modalities, that are synergistic in that they have stronger effects than the sum of the effects of the individual components, and also synergistic with conventional clinical treatments of inflammatory conditions. “Synergistic” is also intended to mean: “coordinated or correlated action by two or more structures or drugs”[Stedman's Medical Dictionary, 23^{rd} ed., Williams & Wilkins, Baltimore, 1976]. An important need also exists for formulations that increase the absorption from the gastrointestinal tract, nasal passages and skin surface of the compositions of the invention. Such formulations have been discovered, and are described below.

### SUMMARY OF THE INVENTION

The invention comprises compositions for human use containing one or more of a flavonoid compound or structurally related analogues, olive kernel extract, hydroxytyrosol, and berberine and, optionally, one or more of a non-bovine sulfated proteoglycan, oleocanthal, S-adenosylmethionine (“SAMe”), a histamine-1 receptor antagonist, a histamine-3 receptor agonist, an antagonist of the actions of CRH, biotin, selenium, emu oil, oregano oil, grape seed oil, a phospholipid, forsynthia fructus extract, or aloe extract. The compositions can also further comprise appropriate excipients and carriers. The compositions of this disclosure have improved absorption from the gastrointestinal tract, skin surface, and nasal and pulmonary surfaces, and anti-inflammatory effects synergistic with each other and synergistic with available conventional clinical treatments.

In some embodiments, the composition further comprises a biologic immune modulator, defined as a drug neutralizing a known inflammatory molecule. Biologic immune modulators include, but are not limited to, T cell inhibitors, TNF inhibitors, and mTOR inhibitors.

In some embodiments, the flavonoid is luteolin (3,3′,4′,5,7-tetrahydroxy flavone), luteolin's structural analogue tetramethoxy luteolin ether, rutin, myricetin, genistein, kaempferol, luteolin, apigenin, (−)-epigallocatechin-3 gallate, kaempferol or the kaempferol glycoside astragaline, hesperitin its glycoside hesperidin, or huperzine A.

In some embodiments, the proteoglycan is non-bovine chondroitin sulfate.

In some embodiments, the composition further comprises one or more phospholipids. In some embodiments, the phospholipid is fish oil, Krill oil, or phosphatidylcholine.

In some embodiments, the compositions can contain antagonists of the actions of CRH on mast cells or other target cells of the myocardium, gastric or intestinal mucosa, urinary bladder, skin, meningeal membranes, blood-brain barrier, and brain structures.

In still another embodiment, the inventive compositions are used for treating or preventing Alzheimer's Disease (AD).

In still another embodiment, the inventive compositions may be used for treating or preventing Autism Spectrum Disorders (ASD).

In another embodiment, the inventive compositions are used for treating or preventing the inflammatory processes of chronic fatigue syndrome (CFS).

In yet another embodiment, the inventive compositions are used for treating fibromyalgia.

In still another embodiment, the inventive compositions may be used in the treatment of the neuroinflammatory aspects of amyotrophic lateral sclerosis.

In another embodiment, the inventive olive kernel extract is used to improve the absorption of biochemicals across membrane barriers in the body, such as those of the intestine, skin, oral mucosa, blood-brain barrier, and pulmonary alveoli.

In yet another embodiment, the inventive compositions may be added to conventional treatments for the aformentioned diseases to increase the anti-inflammatory effects.

### BRIEF DESCRIPTION OF THE FIGURES

FIG. 1 shows the results of a comparison of the inhibitory activity of luteolin (“Lut”) and tetramethoxyluteolin (“Me-lut”) on human mast cell secretion of the pro-inflammatory cytokine tumor necrosis factor (TNF).

FIG. 2 is a diagram showing signaling networks involving PI3K/AKT/mTORC1 and NF-κB in keratinocyte and mast cell activation.

### DETAILED DESCRIPTION OF THE PREFERRED EMBODIMENTS OF THE INVENTION

It has been discovered that compositions comprising a flavonoid, olive kernel extract, hydroxytyrosol, and berberine have synergistic anti-inflammatory effects when used in the treatment of neuro-inflammatory disorders. The compositions also optionally comprise a sulfated proteoglycan, oleocanthal, a CRH antagonist, a histamine-1 receptor antagonist, a histamine-3 receptor agonist, biotin, selenium, emu oil, oregano oil, grape seed oil, forsynthia fructus extract, aloe extract, and S-adenosylmethionine. The compositions can be administered orally, as a topical or transdermal product, or as an aerosol for nasal or pulmonary administration. The compositions can also be used in conjunction with conventional clinical treatments for inflammatory diseases.

Within the present context, inflammatory diseases involve the activation, degranulation and consequent secretion of inflammatory or neurotoxic biochemicals from mast cells. The resultant neuro-inflammatory diseases include, but are not limited to, allergies, Alzheimer's disease (AD), atherosclerosis, asthma, Autistic Spectrum Disorders (ASD), chronic fatigue syndrome, chronic prostatitis, chronic urticaria, coronary artery disease, eczema, fibromyalgia, inflammatory bowel disease, interstitial cystitis, irritable bowel syndrome, ischemia, migraines, mastocytosis, mast cell activation disorder, minimal cognitive impairment, multiple sclerosis, neurofibromatosis, oral inflammation, periodontal disease, peripheral neuralgia, oral inflammation, pruritus (itching), psoriasis, rheumatoid arthritis, rhinitis, superficial vasodilator flush syndromes, temporomandibular joint disorder, and trigeminal neuralgia. The olive kernel extract may be used to improve the transmembrane transport of difficulty-absorbable biomolecules in the brain intestine, skin and pulmonary alveoli.

The term “treating” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The terms “effective amount” are used interchangeably in this disclosure and refer to an amount of a compound that, when administered to a subject, is capable of improving a symptom of a disorder in a subject. The actual amount which comprises the “effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

The terms “administer”, “administering”, or “administration” as used in this disclosure refer to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body

In one embodiment, the sulfated proteoglycan is non-bovine chondroitin sulfate. In other embodiments, the sulfated proteoglycan is keratan sulfate, dermatan sulfate, or hyaluronic acid sodium salt (sodium hyaluronate).

In one embodiment, the flavonoid is luteolin which inhibits secretion of inflammatory molecules from mast cells and mast cell-induced T cell activation. In addition to luteolin, other flavonoids suitable in carrying out the invention include: luteolin's structural analogue tetramethoxy luteolin, quercetin, the quercetin glycoside rutin, myricetin, genistein, apigenin, (−)-epigallocatechin-3 gallate, kaempferol and the kaempferol glycoside astragaline, hesperitin and its glycoside hesperidin, as well as huperzine A.

The compositions comprise berberine. Berberine has been reported to inhibit microglial activation and subsequent inflammation triggered by amyloid-beta associated with the pathogenesis of AD [Jia et al., J Pharm Pharmacol 64:10 (2012)] and oregano oil [Lindsay et al., Br J Nutr 104:513 (2010)].

In some embodiments, the olive kernel extract product is an unrefined (first pressing, filtered, oleic acid-related acidity <0.1%, water content <0.1%) extract produced, for one source MINERVA SA. Edible Oil Enterprises, 31 Valaoritou St., Metamorphosis, Attica, Greece. This kernel extract product is especially prepared by applicant's process consisting of: (1) harvesting first collection ripe olives, preferably in early December; (2) compressing the oil from the flesh of the ripe olives; (3) washing the kernels remaining after step (2) with water to remove debris; (4) drying the washed kernels olive skin and any leaves with a stream of hot air; (5) crushing the dried pomace to produce an extract; (6) extracting the extract from step; (7) removing particulate matter from the organic extract by centrifugation or microfiltering through 1-2 micron pore size filters; (8) evaporating any organic solvent and water from the clarified extract of step (7) by maintaining the extract at about 80 degrees C. while percolating an intert gas (e.g helium to avoid oxidation) through the fluid, which process reduces and (8) storing the final kernel extract product in the absence of air.

The inventive olive kernel extract surprisingly has the unique property of increasing absorption of the other components of the anti-inflammatory compositions through the intestinal mucosa or skin, and also adds its own content of important anti-oxidants, such as omega fatty acids (e.g., eicosapentanoic acid) and alpha tocopherol. The polyphenols found in such olive kernel extracts also have anti-inflammatory effects in, for example, arthritis [Martinez-Dominguez, et al., Inflamm. Res. 50:102 (2001)].

In addition to its usefulness in increasing the absorption of the inventive macromolecular compositions across the intestinal wall and the skin, the inventive olive kernel extract product is useful in decreasing permeability of the blood-brain-barrier [Mohagheghi et al., Scientific World Journal 10:1180 (2010)] and increasing cognition [Berr et al., Dement Geriatr. Cogn. Disord. 28:357 (2009)].

In some embodiments, the compositions of this disclosure further comprise S-adenosylmethionine (“SAMe”), which adds antioxidant, anti-inflammatory and cytoprotective properties, particularly in inflammatory joint and cardiovascular diseases. The addition of SAMe also accelerates metabolism of homocysteine, which amino acid has been implicated in coronary artery disease, to cysteine, which is harmless. Folic acid or its more water soluble folinic acid may also be added to certain of the present formulations for similar reasons.

In some embodiments, the compositions of this disclosure further comprise a histamine-1 receptor antagonist, such as hydroxyzine, merelastine, azelastine, azatadine, rupatadine, and cyproheptadine. Other histamine-1 receptor antagonists are described in Table 25-1 in Goodman and Gilman's The Pharmaceutical Basis of Therapeutics, 9^{th} ed., New York, 1996.

In additional embodiments, the compositions of this disclosure further comprise a histamine-3 receptor agonist, such as R(−)-α-methyl histamine, N^{α}-methyl histamine, N′-methyl histamine, α-N^{α}-dethylhistamine, α,β-dimethyl histamine, N^{α}-methyl-α-(dimethyl)histamine, N^{α}-methyl-α-(chloromethyl)histamine, or α,β-difluoro-N^{α}-(fluoromethyl)histamine. Histamine-3 receptor agonists are also described in the patents listed above.

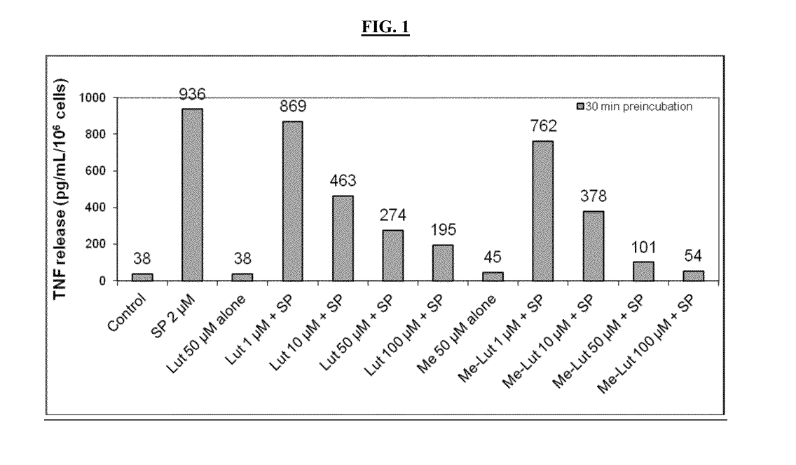

Flavonoids are reported to reduce the severity of experimental autoimmune encephalomyelitis in mice, a model for multiple sclerosis [Verbeck, et al., Biochem. Pharm. 70(2):220 (2005); Hendriks, et al., J Exp Med. 200(12):1667 (2004)], as well as that of AD [Parker-Anthill et al., J Neuroimmunol 217:20 (2009) ] and ASD [Theoharides et al., Int J Immunopathol Pharmacol 25:317 (2012)]. An unexpected finding is that tetramethoxy luteolin ether, a structural analog of luteolin the four hydroxy groups of which luteolin have been replaced with a methyl group, has higher ability to penetrate into cells, has better oral absorption, and exhibits stronger inhibition of the release of the potent inflammatory cytokine tumor necrosis factor (TNF) from activated human mast cells (Example 1/FIG. 1).

Sources of CRH antagonists include, in addition to the patents listed above: Neurocrine Biochem. Inc.'s D-Phe 12 NIe A1a32,21,38hCRH(12-41)NR2, cat no. 1P-36-41; Pfizer non-peptide CP-154,526-1; Sigma Chem., St. Louis anti-CRH polyclonal antiserum; and Pfizer, NY patents and applications: U.S. Pat. No. 6,211,195, U.S. Pat. No. 5,795,905, PCT/IB95/00573, PCT/IB95/00439, U.S. Ser. No. 08/448,539, U.S. Ser. No. 08/481,413, U.S. Ser. No. 09/735,841, and in Owens et al. Pharm. Rev. 43:425 (1991).

The preferred concentration range of the flavonoid and proteoglycan components of the oral formulations are 100-3,000 mg per unit dose. Generally, where present, the amount of the olive kernel extract is at about 50% of the other ingredients. The number of capsules, pills or tablets to be taken per day or skin cream or ointment to be applied is determined by the nature and severity of the disease, and is readily determinable by the patient's health provider. Other representative formulations are described in the examples below.

The compositions of the invention may be formulated in any standard means of introducing pharmaceuticals into a patient, e.g., by means of capsules, tablets or pills, longenes, subliungual, transdermal or suppository preparations, in either powder, phospolipid vesicle (liposome), or phosphatidyl choline mixture. The compositions of the invention also include ointments, creams for skin conditions, mouth washes and toothpaste for oral inflammation and solutions for nasal aerosols. Standard excipients and carriers for the active ingredients of the inventive compositions are described in Remington's Pharmaceutical Sciences, Mack Publishing Co., Easton, Pa.

Although not bound by any particular mechanism of action of the claimed compositions, it is believed that they inhibit the activation of mast cells in the affected tissues or organs, and inhibit the release of inflammatory or neurotoxic biomolecules from these mast cells. “Activation” is heretofore considered synonymous to “release” or “secretion” from the activated mast cells through degranulation vesicles, exosomes, traps or tunneling nanotubules of any type of molecule(s) that alone or in combination triggers or contributes to inflammation or neuro-inflammation either directly or through subsequent stimulation of other immune cells, including macrophages, microglial cells or T cells.

### EXAMPLES

### Example 1

FIG. 1 shows the comparison of the inhibitory activity of luteolin (“Lut”) and tetramethoxyluteolin (“Me-lut”) on human mast cell secretion of the pro-inflammatory cytokine tumor necrosis factor (TNF). Human cultured mast cells were incubated at 37° C. in a humidified 95% O₂/5% CO₂ incubator either alone (control), stimulated by SP, or pretreated with either luteolin or methoxyluteolin alone for 30 min without any stimulation, or pretreated with different flavonoid concentration followed by stimulation with SP as shown. TNF was assayed in the supernatal fluid by Enzyme-linked immunosorbent assay (ELISA).

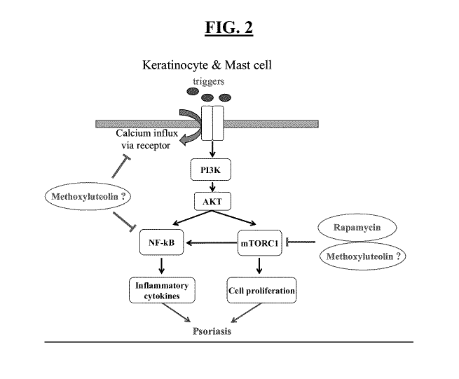

FIG. 2 is a diagram showing signaling networks involving PI3K/AKT/mTORC1 and NF-κB in keratinocyte and mast cell activation. Stimulation by triggers such as TNF or NT leads to PI3K activation, which recruits and subsequently activates Akt. Activated PI3K/Akt phosphorylates mTOR and induces cell proliferation. Phosphorylated mTOR also activates NF-κB nuclear translocation, leading to inflammatory cell proliferation and pro-inflammatory cytokine production and inflammation. Rapamycin blocks the over-active mTOR activity. The flavonoid methoxyluteolin may also act by effectively blocking mTOR activity and potentially other steps in the signaling network. This blocking leads to a disruption in the signaling steps important for inflammation.

### Example 2

The kinase activity of mTOR is inhibited by the antibiotic rapamycin (Ehninger, Trends Mol. Med 17:(2), 2011; Sahin, Curr Opin Neurobiol. 2012). However, there has been intense effort to discover additional molecules that may effectively block this pathway. The flavonoid quercetin was reported to inhibit Akt/mTOR signaling in gastric cancer decreasing cellular proliferation {Wang, 2011 19556 /id}. In addition, the major green tea flavonoid epigallocatechin-3-gallate (EGCG) is a dual inhibitor PI3K/mTOR pathway (Van Aller et al. Biochem Biophys Res Commun. 406:(2), 2011). Finally, combination of rapamycin and isoflavones was shown to have greater inhibition of mTOR pathway in human glioblastoma cells (Puli et al. Neurochem Res. 35:(7) 2010).

Methoxyluteolin, a structural analogue of luteolin, was developed and showed better cell penetration. Evidence indicated that methoxyluteolin can inhibit multiple signaling steps important in inflammation especially mTOR (FIG. 2). Methoxyluteolin based local or systemic formulations thus can be used in the treatment of inflammatory diseases.

### Example 3

Composition For Protecting Against Mastocytosis

| Ingredients | mg/unit dose |
|---|---|
| Tetramethoxyluteolin | 150-500 |
| Quercetin | 150-500 |
| Olive kernel extract | 300-1000 |

### Example 4

Composition For Protecting Against Chronic Fatigue Syndrome

| Ingredients | mg/unit dose |
|---|---|
| Co-enzyme Q10 | 150-300 |
| Luteolin | 150-300 |
| Carnitine | 100-200 |
| Quercetin | 150-300 |
| Olive kernel extract | 300-1200 |

### Example 5

Composition For Protecting Against Fibromyalgia

| Ingredients | mg/unit dose |
|---|---|
| Chondroitin sulfate | 150-300 |
| Carnitine | 100-300 |
| White willow bark extract | 50-150 |
| Quercetin | 150-300 |
| Co-enzyme Q10 | 150-300 |
| Olive kernel extract | 300-1200 |

### Example 6

Composition For Protecting Against Coronary Artery Disease

| Ingredients | mg/capsule: |
|---|---|
| Quercetin | 500-1000 |
| S-adenosylmethionine | 50-100 |
| Niacin (slow release)* | 50-500 |
| Olive kernel extract | 50-500 |
| Bitter willow bark extract | 50-150 |
| Olive Kernel Oil | 300-1200 |

*Slow release niacin may be given separately

### Example 7

Composition For Protecting Against oral Inflammatory
Disease (Mouthwash)

| Ingredients | mg % by weight |
|---|---|
| Chondroitin sulfate | 50-150 |
| Tetramethoxyluteolin | 10-100 |

*In a standard mouthwash vehicle

### Example 8

Composition For Protecting Against oral
Inflammatory Disease (Toothpaste)

| Ingredients | mg % by weight |
|---|---|
| Chondroitin sulfate | 5-50 |
| Tetramethoxyluteolin | 10-100 |
| Olive kernel extract | 10-150 |

### Example 9

Composition For Protecting Against Skin Inflammation

| Ingredients | mg % by weight |
|---|---|
| Tetramethoxyluteolin | 10-100 |
| Forsynthia fructus extract | 10-100 |
| Aloe vera extract | 10-100 |
| Olive kernel extract | 50-350 |
| Emu oil | 50-350 |

### Example 10

Oral Composition For Protecting Against Migraine Headaches

| Ingredients | mg/unit dose |
|---|---|
| Tetramethoxyluteolin | 100-1000 |
| Optionally, a CRH-receptor antagonist, or | |
| Azatidine or Cyproheptadine | |

### Example 11

Oral Composition For Protecting Against in Relapsing Multiple Sclerosis

| Ingredients | mg/unit dose |
|---|---|
| Quercetin | 50-300 |
| Tetramethoxyluteolin | 10-1000 |
| Hydroxyzine | 50-300 |
| Olive kernel extract | 350-1200 |
| Optionally, glatiramer acetate, or interferon-beta | |

### Example 12

Composition For Protecting Against Cystitis And Prostatitis

| Ingredients | mg/unit dose |
|---|---|
| Berberine | 100-300 |
| Chondroitin sulfate | 100-300 |
| Sodium hyaluronate | 50-200 |
| Quercetin | 100-400 |
| Olive kernel extract | 350-1200 |

### Example 13

Composition For Protecting Against Niacin-induced“Flush”*

| Ingredients | mg/per unit dose |
|---|---|
| Luteolin | 100-1000 |
| Quercetin | 500-1000 |
| Olive kernel extract | 100-3000 |
| White willow bark extract | 10-500 |

### Example 14

Topical Composition For Protecting Against Skin Allergic Inflammation

| Ingredients: | mg % by weight |
|---|---|
| Tetramethoxy luteolin ether | 10-100 |
| Quercetin | 10-200 |
| Forsynthia fructus extract | 10-200 |
| Emu oil | 10-100 |
| Olive kernel extract | 10-1000 |
| Aloe extract | 10-500 |
| Oregano oil | 1-10 |

### Example 15

Composition For Protecting Against Inflammation Allergic and Asthma

| Ingredients | mg/day |
|---|---|
| Tetramethoxy luteolin | 50-500 |
| Forsynthia fructus extract | 10-20 |
| Quercetin | 50-500 |
| Olive Kernel extract | 5-100 |

### Example 16

Composition For Protecting Against Minimal Cognitive Impairment
or Alzheimer's Disease, or Dementia

| Ingredients | mg/day |
|---|---|
| Tetramethoxyluteolin | 50-300 |
| Luteolin | 50-300 |
| Hydroxytyrosol | 50-300 |
| Oleocanthal | 50-300 |
| Berberine | 50-300 |
| Folinic acid | |
| Selenium | |
| Olive Kernel extract | 300-1000 |

### Example 17

Composition For Protecting Against Autism Spectrum Diseases

| Ingredients: | mg/day |
|---|---|
| Tetramethoxyluteolin | 100-1000 |
| Luteolin | 100-1000 |
| Berberine | 50-500 |
| Olive Kernel Extract | 100-1000 |

### Example 18

### Effect of Olive Kernel Extract on Absorption of a Proteoglycan Sulfate In Vivo

Chondroitin sulfate was tritiated by New England Nuclear Corp. to a specific activity of 4.3 mCi/ml.

Unlabeled chondroitin sulfate was dissolved in olive kernel extract at a ratio of about 55 w/v chondroitin sulfate powder to about 450 w/v of olive kernel extract (2.9% acidity as oleic acid, 1.03% water, 0.08% hexane). To this solution was added 20.2 microCuries of the labeled chondroitin sulfate and the suspension was mixed using a Vortex AAA gelatin capsules were filled with the resulting solution using an aluminum template molding device.

The laboratory animals (250 g male Sprague-Dawley rats) were kept overnight without food but with free access to water. One capsule containing the above-described chondroitin sulfate-olive kernel extract solution was given to each rat per os. Control animals were given the equivalent amount of chondroitin sulfate, but without olive kernel extract. The animals were then given free access to food. Blood was obtained from trail veins and serum radioactivity was measured 8 hours thereafter in a beta scintillation counter.

The results showed that, in control animals, about 3.9%+/−0.4% (n=3) of the dose of labeled chondroitin sulfate reached the circulation. In sharp contrast, in animals given the olive kernel extract containing the labeled chondroitin sulfate, about 14.3%+/−0.7% (n=4) of the dose was absorbed into the general circulation.

These results demonstrate that olive kernel extract increased the absorption of a proteoglycan from the intestine into the general circulation by almost 400%.

### Example 18

Composition For Protecting Against Autism Spectrum
Diseases, NeuroProtek ®

| Ingredients: | mg/day |
|---|---|
| Luteolin | 100-1000 |
| Quercetin | 50-500 |
| Rutin | 1-50 |
| Olive Kernel Extract | 100-1000 |

### Example 19

A 7 year old girl with ASD started talking NeuroProtek® in November 2010. In January 2011, her pediatrician reported he was stunned by the difference. She started greeting people, and started engaging in play with her preschool class friends. The school also noticed major improvement with her self regulations and modulation.

### Example 20

A boy with ASD had been on NeuroProtek® for 4 months. He started communicating better. His test scores and homework showed incredible improvement. His school teachers noticed the difference, and the leader from the Boy Scouts noted that his focus, understanding and social skills have changed drastically within the last couple of weeks. The boy managed to write his first story ever and read it at the school Christmas function in 2011.

### Example 21

One 7 year old boy with ASD has been taking NeuroProtek® for several weeks now, and already there have been noticeable and very positive changes in several areas, especially in cognition and academic performance.

### Example 22

The mother of a 5 year old boy with autism mentioned that her son was on NeuroProtek® for 6 months and showed great improvement. He learned how to bicycle, how to use a laptop, how to swim and all these with little effort. He became more sociable and people around him noticed a different towards the better.

### Example 23

Composition For Protecting Against Minimal Cognitive Impairment
(“Brain Fog”), NeuroActif ®

| Ingredients: | mg or microg/day | |
|---|---|---|
| Berberine | 50-500 | |
| Biotin | 1-10 | mg |
| Folinic acid | 100-1000 | microg |
| Hydroxytyrosol | 200-2000 | mg |
| Luteolin | 100-1000 | |
| Oleocanthal | 50-100 | |
| Olive Kernel Extract | 100-1000 | |
| Selenium | 10-100 | microg |

### Example 24

A 37 year old white female with the diagnosis of mast cell activation syndrome complained of fatigue, “brain fog”, lack of concentration and declining short term memory for the last two years. She started on NeuroActif® (4 capsules per day) two months ago and during a meeting over last weekend reported significant improvement of all symptoms.

### Example 25

A 53 year old white male with the diagnosis of chronic fatigue syndrome was almost debilitated by “brain fog”, lack of concentration and rapidly declining short term memory for the last seven months to the point of leaving his computer software analyst job. He had tried Aricept and Ginkgo biloba for many months without any benefit. He started on NeuroActif® (4 capsules per day) three months ago and is now almost clear of all symptoms.

### Example 26

A 29 year old white female with no clear diagnosis, but multiple endocrine and allergic symptoms has been complaining of chronic fatigue, “brain fog”, lack of concentration and declining short term memory for the seven years. She had tried multiple medications and natural supplements without any benefit. She started on NeuroActif® (4 capsules per day) one month ago and is already reporting significant improvement.

### Example 27

A 62 year old white woman has had fibromyalgia accompanied by brain fog”, lack of concentration and poor short term memory for at least five years. She had been prescribed antidepressants, L-carnitine, and a variety of pain killers without much benefit. She has been on NeuroProtek® (4 capsules per day) for the last year with almost compete resolution of her “brain fog.”

1. A method of treating a neuro-inflammation disorder in a subject, comprising administering to a subject in need thereof an effective amount of a composition comprising a flavonoid, or a structurally related analogue, olive kernel extract, hydroxytyrosol, and berberine, and, optionally, one or more ingredients selected from the group consisting of a sulfated proteoglycan, oleocanthal, a CRH antagonist, S-adenosylmethionine, a histamine-1 receptor antagonist, a histamine-3 receptor agonist, emu oil, oregano oil, grape seed oil, aloe extract, biotin, huperzine A, and selenium.

2. The method of claim 1, wherein the composition further comprises a biologic immune modulator.

3. The method of claim 2, wherein the biologic immune modulator is a T cell inhibitor.

4. The method of claim 2, wherein the biologic immune modulator is a TNF inhibitor.

5. The method of claim 2, wherein the biologic immune modulator is an mTOR inhibitor.

6. The method of claim 1, wherein the flavonoid is selected from the group consisting of luteolin, tetramethoxyluteolin, quercetin, myricetin, genistein, kaempferol, (−)epigallocatechin-3-gallate, epigenin, rutin, hesperitin, hesperidin, and huperzine A.

7. The method of claim 1, wherein the composition further comprises a phospholipid, and wherein the phospholipid is selected from the group consisting of fish oil, Krill oil, or phosphatidylcholine.

8. The method of claim 1, wherein the composition comprises luteolin, tetramethoxyluteolin, quercetin, olive kernel extract, hydroxytyrosol, oleocanthal, berberine, biotin, and selenium.

9. The method of claim 8, wherein the composition further comprises hydroxyzine.

10. The method of claim 8, wherein the composition is administered orally.

11. The method of claim 8, wherein each ingredient is in the amount of about 1-1,000 mg per unit dose.

12. The method of claim 8, where the composition comprises 10-1000 mg of luteolin, 10-1000 mg of tetramethoxyluteolin, 100-1000 mg of hydroxytyrosol, 100-1000 mg of oleocanthal, 10-1000 mg berberine, and 10-1000 mg of olive kernel extract.

13. The method of claim 12, wherein the composition comprises of 100 mg luteolin, 100 mg tetramethoxyluteolin, 50 mg of hydroxytyrosol, 50 mg of berberine, and 400 mg of olive kernel extract.

14. The method of claim 13, wherein the composition is administered orally.

15. The method of claim 1, wherein the composition is administered topically.

16. The method of claim 15, wherein in the composition is a cream, a lotion, or a form capable of transdermal administration.

17. The method of claim 16, wherein the composition comprises chamomile extract, tetramethoxyluteolin, olive kernel extract, emu oil, oregano oil, aloe extract, and forsynthia fructus extract.

18. The method of claim 17, wherein the composition comprises by weight 5% chamomile extract, 5% teramethoxyluteolin, 1% emu oil, 0.01% oregano oil, 20% aloe extract, 5% forsynthia fructus extract, and balanced olive kernel extract.

19. The method of claim 1, wherein the composition is formulated for intranasal administration.

20. The method of claim 19, wherein the composition comprises of tetramethoxyluteolin , olive kernel extract, and forsynthia fructus extract.

21. The method of claim 1, wherein the neuro-inflammatiory disorder is selected from the group consisting of Alzheimer's disease, allergies, atherosclerosis, asthma, Autistic Spectrum Disorders, chronic fatigue syndrome, chronic prostatitis, chronic urticaria, coronary artery disease, eczema, fibromyalgia, inflammatory bowel disease, interstitial cystitis, irritable bowel syndrome, ischemia, migraines, mastocytosis, mast cell activation disorder, minimal cognitive impairment, multiple sclerosis, neurofibromatosis, pruritus, oral inflammation, periodontal disease, peripheral neuralgia, psoriasis, rheumatoid arthritis, rhinitis, superficial vasodilator flush syndromes, temporomandibular joint disorder, and trigeminal neuralgia.
